(19) United States
(12) Patent Application Publication
(10) Pub. No.: US 2010/0113286 A1
(43) Pub. Date: May 6, 2010

(54) METHODS FOR DETECTION OF PREECLAMPSIA

(76) Inventors: Gilles Andre Lajoie, Waterloo (CA); Victor Khin Maung Han, London (CA); Aaron Timothy Booy, Innisfil (CA)

Correspondence Address: VALENTINE A COTTRILL;SUSAN TANDAN, 50 QUEEN STREET NORTH, STE. 1020, P.O. BOX 2248, KITCHENER, ON, N2H6M2, CA

(21) Appl. No.: 12/451,296

(22) PCT Filed: May 5, 2008
(86) PCT No.: PCT/CA2008/000852

§ 371 Date: Jan. 6, 2010

Related U.S. Application Data
(60) Provisional application No. 60/927,732, filed on May 5, 2007.

(51) Int. Cl. C40B 30/00; C12Q 1/00; C12Q 1/32; C40B 40/04

(52) U.S. Cl. 506/7; 435/4; 435/26; 506/15

(57) ABSTRACT

Biomarkers associated with preeclampsia were identified. Nine proteins were identified as being differentially regulated between the control and the under 28-weeks preeclamptic samples. Similarly three proteins were identified as being differentially regulated between the control and the over 28-weeks preeclamptic samples. These 12 proteins can be used as potential biomarkers in the diagnosing and prognosing of preeclampsia.

### FIELD OF THE INVENTION

The present invention generally relates to the detection of preclampsia, and more particularly, to biomarkers useful in the detection of preclampsia.

### BACKGROUND

Preeclampsia is a pregnancy specific disease that affects 5-8% of all pregnancies worldwide, and is one of the leading causes of maternal and neonatal morbidity and mortality. The term “preeclampsia” refers to the development of elevated blood pressure and protein in the urine after the 20th week of pregnancy, associated symptoms and related disorders. While the cause of preeclampsia is still unknown, clinical evidence unequivocally points to the placenta as the site of pathophysiology. It is widely accepted that impaired cellular invasion during placental development and a lack of proper development of the decidual spiral arteries account for the development of preeclampsia. Despite considerable research, the factor(s) that ultimately cause preeclampsia have not been fully characterized.

Currently, a combination of various general tests (e.g. blood pressure, protein concentration in blood, etc.), and other physical conditions leads a physician to an eventual diagnosis. However, proving that a pregnancy is preeclamptic once the symptoms are fully developed in the third trimester is of little use for prophylactic clinical screening. At present, there is no accurate method for the early detection of preeclampsia.

Accordingly, it would be desirable to identify biomarkers of preeclampsia that may be used to detect preeclampsia before symptoms develop in order that the condition may be appropriately treated.

### SUMMARY

Differentially expressed proteins in preeclampsia have now been identified that are useful as biomarkers for diagnosing, prognosing and staging, predicting the onset of and treating preeclampsia. Many of these proteins are functionally related and point to several biological pathways or processes involved in the pathology of preeclampsia.

Accordingly, in one aspect of the invention, a method for diagnosing, or detecting or predicting the onset of preeclampsia in a mammal is provided. The method comprises the steps of:

(a) determining in a biological sample from the mammal the expression level or activity of at least one preeclampsia biomarker, and

(b) comparing the expression level or activity of each preeclampsia biomarker with the level or activity of the biomarker in a healthy mammal free of preeclampsia, wherein a differential expression level or differential activity of the biomarker is indicative of preeclampsia in the mammal, or that preeclampsia may develop in the mammal.

In another aspect of the invention, a method of prognosing, monitoring or staging preeclampsia in a mammal is provided, comprising the steps of:

(a) determining in a first biological sample from the mammal the expression level or activity of at least one preeclampsia biomarker, and

(b) comparing the expression level or activity of each preeclampsia biomarker with the level or activity of the biomarker in the mammal as determined in a second biological sample obtained prior to the first sample to monitor the preeclampsia in the mammal.

In a further aspect, a microarray for use in diagnosing preeclampsia in a mammal is provided, comprising at least one reagent capable of detecting at least two biomarkers of preeclampsia exhibiting a differential level of expression or level of activity in a mammal having preeclampsia in comparison with the level exhibited in a healthy mammal.

In a further aspect, a method of screening candidate therapeutic compounds for treating preeclampsia in a mammal is provided. The method comprises the steps of:

(a) incubating a biological sample that expresses at least one preeclampsia biomarker with said candidate therapeutic, and

(b) determining whether said candidate therapeutic modulates the expression or activity of the preeclampsia biomarker, wherein modulation of the level of expression or activity of the preeclampsia biomarker indicates that the candidate therapeutic may be a therapeutic agent for treating or preventing preeclampsia.

In another aspect of the invention, a method of monitoring the treatment of preeclampsia in a mammal is provided, comprising:

(a) determining in a biological sample from the mammal the expression level or activity of at least one preeclampsia biomarker at different time points during treatment; and

(b) comparing the expression level or activity of each preeclampsia biomarker at each time point with the level or activity of the biomarker in a healthy mammal, wherein a reduction in the differential expression level or differential activity of the biomarker in the mammal over time as compared with the healthy mammal is indicative that the treatment is effective.

In yet another aspect of the invention, a kit useful for the diagnosis of preeclampsia is provided. The kit comprises at least one reagent that is useful to detect a biomarker that exhibits differential expression or activity in a biological sample from a mammal with preeclampsia in comparison with the expression or activity thereof in a healthy mammal.

These and other aspects of the present invention will become apparent from the description, drawings, and claims that follow.

### BRIEF DESCRIPTION OF THE DRAWINGS

FIG. 1 depicts the functional classification of the proteins identified as being differentially expressed in either the over or under-28 week preeclampsia discovery sample sets.

### DETAILED DESCRIPTION

The present invention is based on the identification of a number of biomarkers useful for the early detection, diagnosis, monitoring, staging, prediction, and prognosis of preclampsia in a mammal. A biomarker useful for the detection of preeclampsia exhibits a differential level of expression or activity in comparison with a healthy mammal and may be identified in a biological sample obtained from a mammal.

The term “a preeclampsia biomarker” as used herein refers to a component of a biological sample obtained from a mammal that exhibits a differential level of expression or activity in comparison with a healthy mammal. The term “healthy mammal” is used herein to refer to a pregnant mammal that does not have preeclampsia nor a predisposition to develop preeclampsia. Examples of preeclampsia biomarkers in accordance with the invention include, but are not limited to, fatty acid binding protein 4 (FABP4), enoyl-CoA hydratase (ECHS1), Δ3,5-Δ2,4-dienoyl-CoA isomerase (ECH1), antioxidant protein Per6, heat shock protein ∃-1, stathmin, lipocortin 1 (LPC1), prostaglandin dehydrogenase 1, proliferation-associated protein 2G4, placental growth hormone (chorionic sommatomammotropin hormone (CSH1), estradiol 17-beta dehydrogenase (HSD17B1) and macrophage capping protein, as well as metabolites and derivatives thereof, including fatty acids, lipids and proteins, which are similarly associated with preclampsia, as one of skill in the art would appreciate.

The term “mammal” is used herein to include both human and non-human mammals such as domestic and wild animals.

The term “FABP4” as used herein refers to mammalian fatty acid binding protein 4 including both human FABP4, as depicted by accession number, (sp|P15090|FABPA_HUMAN), as well as non-human equivalents thereof.

The term “ECHS1” refers to mammalian enoyl-CoA hydratase including both human ECHS1 as depicted by accession number, sp|P30084|ECHM_HUMAN), as well as non-human equivalents thereof.

The term “ECH1” refers to mammalian Δ3,5-Δ2,4-dienoyl-CoA isomerase including both human ECH1, as depicted by accession number sp|Q13011|ECH1_HUMAN), and non-human equivalents thereof.

The term “Per6” refers to mammalian antioxidant protein Per6 including both human Per6, as depicted by accession number, sp|P30041|PRDX6_HUMAN), as well as corresponding non-human equivalents thereof.

The term “heat shock protein ∃-1” refers to mammalian forms of the protein including the human form, as depicted by accession number sp|P04792|HSPB1_HUMAN, as well as corresponding non-human equivalents thereof.

The term “stathmin” refers to mammalian stathmin including both human stathmin as depicted by accession number, sp|P16949|STMN1_HUMAN), as well as corresponding non-human equivalents thereof.

The term “prostaglandin dehydrogenase 1” refers to mammalian forms of the protein including the human form, as depicted by the accession number sp|P15428|PGDH_HUMAN, as well as corresponding non-human equivalents thereof.

The term “LPC1” refers to mammalian lipocortin including both human LPC1 as depicted by accession number, (sp|P04083|ANXA1_HUMAN), and corresponding non-human equivalents thereof.

The term “2G4” refers to mammalian proliferation-associated protein 2G4 as depicted by accession number, (sp|Q80|PA2G4_HUMAN), as well as corresponding non-human equivalents thereof.

The term “CSH1” refers to human placental growth hormone (chorionic sommatomammotropin hormone) as depicted by accession number, (sp|P01243|CSH_HUMAN), as well as corresponding non-human equivalents thereof.

The term “HSD17B1” refers to human estradiol 17-beta dehydrogenase as depicted by accession number, sp|P14061|DHB1_HUMAN), as well as corresponding non-human equivalents thereof.

The term “macrophage capping protein” refers to mammalian forms of the protein including the human form as depicted by accession number, sp|P40121|CAPG_HUMAN, as well as corresponding non-human equivalents thereof.

Preclampsia biomarkers in accordance with the invention may be involved in certain metabolic functions. In one embodiment, preeclampsi biomarkers are involved in the metabolism of fatty acids, for example, fatty acid binding protein 4 (FABP4), enoyl-CoA hydratase (ECHS1), and Δ3,5-Δ2,4-dienoyl-CoA isomerase (ECH1). In another embodiment, preeclampsia biomarkers have a function related to the metabolism of estrogen precursors, for example, estradiol 17-beta dehydrogenase and proliferation-associated protein 2G4 (PA2G4), and in another embodiment, preeclampsia biomarkers are related to oxidative stress, such as the antioxidant protein Per6, heat shock protein β-1 (HSP27) and stathmin. Lipocortin (LPC1) and prostaglandin dehydrogenase 1 (PDGH1) preeclampsia biomarkers are involved in the inflammatory response, and proliferation-associated protein 2G4, CSH1, estradiol 17-beta dehydrogenase (HSD17B1) and macrophage capping protein (CAPG) are involved in growth control and regulation.

Preclampsia biomarkers in accordance with the invention include proteins, polynucleotides encoding the proteins, and metabolites or derivatives thereof, that are up-regulated, i.e. exhibit a greater expression level, or activity, in a mammal with preeclampsia than the expression or activity level normally exhibited in a healthy mammal. Examples of preclampsia biomarkers that are up-regulated include, for example, fatty acid binding protein 4 (FABP4), the antioxidant protein Per6, heat shock protein ∃-1, prostaglandin dehydrogenase 1, and macrophage capping protein.

Preclampsia biomarkers in accordance with the invention also include proteins, polynucleotides encoding them, and metabolites or derivatives thereof, that are down-regulated, i.e. exhibit a reduced expression level, or activity, in mammals with preeclampsia than the expression or activity level normally exhibited in healthy controls. Examples of preclampsia biomarkers that are down-regulated include proliferation-associated protein 2G4 (PA2G4), enoyl-CoA hydratase (ECHS1), Δ3,5-Δ2,4-dienoyl-CoA isomerase (ECH1), stathmin, lipocortin (LPC1), proliferation-associated protein 2G4, placental growth hormone or chorionic sommatomammotropin hormone (CSH1) and estradiol 17-beta dehydrogenase (HSD17B1).

In the present method for diagnosing preeclampsia in a mammal, the level of expression or activity of at least one preeclampsia biomarker in a biological sample of a mammal is detected. The term “biological sample” is used herein to refer to any preeclampsia biomarker-containing sample that is obtainable from a mammal. In one embodiment, it is preferred that the sample is one which may be obtained non-invasively including, for example, saliva, sputum, urine, sweat, cervical secretion and vaginal or cervicovaginal mucous. In another embodiment, and where the preeclampsia biomarker is a protein, the biological sample may be a nucleic acid-containing sample suitable to detect nucleic acid encoding the preeclampsia biomarker including, for example, saliva, urine and other bodily secretions, as well as hair, epithelial cells and the like. Although such non-invasively obtained biological samples are preferred for use in the present method, one of skill in the art will appreciate that invasively-obtained protein and nucleic acid-containing biological samples may also be used in methods according to the invention, including for example, blood including white blood cells, serum, plasma, amniotic fluid, placental tissue and standardized placental villi, bone marrow, peritoneal fluid, pleural fluid, cerebrospinal fluid (CSF) and lymph node samples. Techniques for the invasive process of obtaining such samples are known to those of skill in the art.

The methods comprise detecting the expression level or activity of at least one preeclampsia biomarker in a biological sample of a subject mammal and comparing that level to a control that corresponds to the level in a healthy mammal. The term “activity” is used herein to refer to the “biological activity”, “bioactivity” or “biological function” of the biomarker. Exemplary activities include inhibiting or activating a bioactivity by binding to another biomolecule, or by otherwise catalyzing the activity of another biomolecule. A bioactivity may be modulated by directly affecting the subject biomarker. Alternatively, a bioactivity may be altered by modulating the level of the polypeptide, such as by modulating expression of the corresponding gene.

The level of expression of a preeclampsia biomarker may be determined by measuring the level of the expressed protein. This may be done, e.g., by immunoprecipitation, ELISA, or immunohistochemistry using an agent, e.g., an antibody, that specifically detects the protein. Other techniques include Western blot analysis. Immunoassays are commonly used to quantitate the levels of proteins in biological samples, and many other immunoassay techniques are known in the art. The invention is not limited to a particular assay procedure, and therefore is intended to include both homogeneous and heterogeneous procedures. Exemplary immunoassays which may be conducted according to the invention include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA).

For use in immunoassays, an indicator moiety, or label group, may be attached to the subject antibodies and is selected so as to meet the needs of various uses of the method which are often dictated by the availability of assay equipment and compatible immunoassay procedures. Exemplary labels include, but are not limited to, labels which when fused to a preeclampsia biomarker molecule produce a detectable fluorescent signal, including, for example, green fluorescent protein (GFP), enhanced green fluorescent protein (EGFP), Renilla reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED). In another embodiment, a preeclampsia biomarker polypeptide is conjugated to a fluorescent or chromogenic label. A wide variety of fluorescent labels are available from and/or extensively described in the Handbook of Fluorescent Probes and Research Products 8^{th} Ed. (2001), available from Molecular Probes, Eugene, Oreg., as well as many other manufacturers. In other embodiments, a preeclampsia biomarker is fused to a molecule that is readily detectable either by its presence or activity, including, but not limited to, luciferase, chloramphenicol acetyl transferase, β-galactosidase, secreted placental alkaline phosphatase, β-lactamase, human growth hormone, and other secreted enzyme reporters.

The activity of a biomarker may also be evaluated to identify the presence of the biomarker in a biological sample. In this case, assays which measure a reaction pathway mediated by or otherwise depending on the activity of the biomarker may be used, which, for example, yields a reaction product that is readily measurable.

The expression level of a preeclampsia biomarker can also be determined by measuring the presence of gene encoding the biomarker. Suitable methods for this purpose include, for example, reverse transcription-polymerase chain reaction (RT-PCR), a “sandwich” hybridization technique in which oligonucleotide probes are used to capture and detect multiple nucleic acid targets (as described in UK Patent Application GB 2156074A, Oct. 2, 1985), dotblot analysis, reverse dotblot analysis, Northern blot analysis and in situ hybridization. Serial analysis of gene expression (SAGE) technique (as described in Velculescu et al. (1995) Science 270, 484-487) may also be used, the advantages of which include the potential to provide detection of all genes expressed in a given cell type, the provision of quantitative gene expression information and the provision of sequence information that may be used to identify the detected genes. Techniques for producing and probing nucleic acids are further described, for example, in Sambrook et al., Molecular Cloning: A Laboratory Manual (New York, Cold Spring Harbor Laboratory, 1989). The level of expression of a preeclampsia biomarker may also be determined by determining its AQUA™ score, e.g., by using the AQUA™ automated pathology system. AQUA™ (for Automated Quantitative Analysis) is a method of analysis of absolute measurement of protein expression in situ.

In embodiments wherein the preeclampsia biomarker is a fatty acid, mass spectrometry in combination with a chromatographic method such as gas chromatography or HPLC or thin layer chromatography may be used to detect the level thereof in a biological sample.

When analyzing from a biological sample including cells or tissue, it may be important to prevent any further changes in gene expression after the tissue or cells has been removed from the mammal. Changes in expression levels are known to change rapidly following perturbations, e.g., heat shock or activation with lipopolysaccharide (LPS) or other reagents. In addition, the RNA and proteins in the tissue and cells may quickly become degraded. Accordingly, in a preferred embodiment, cells obtained from a mammal are snap frozen as soon as possible.

Detection of a differential level of expression or activity of one or more preeclampsia biomarkers from the control expression level or activity as determined in a healthy mammal is indicative of preeclampsia, or a particular stage of preeclampsia. The phrase “differential level of expression or activity” as it is used herein with respect to preeclampsi biomarkers refers to a change or deviation in the expression or activity of the biomarker in comparison to a control level as determined in a healthy mammal. The amount of deviation in the expression or activity of the preeclampsia biomarkers is not restricted with respect to their utility as a preeclampsia biomarkers. The degree or severity of preeclampsia may be determined based on the degree of deviation in the expression level or activity of the preeclampsia biomarker in a mammal as compared to a control. For example, a mammal exhibiting a greater deviation in the expression level or activity of the preeclampsia biomarker as compared to a control may indicate that the mammal is preeclamptic, is more susceptible to preeclampsia, or is suffering from a more severe case of preeclampsia. On the other hand, a lesser deviation in the expression level or activity of the preeclampsia biomarker as compared to a control may be a very early indication of preeclampsia, or may indicate that a preeclamptic condition is improving.

The above-described method may comprise comparing the determined level of expression of one or more preeclampsia biomarkers with a control expression level which is the level of the biomarker in a healthy mammal, or with a reference set of expression or activity levels of the preeclampsia biomarkers to determine whether or not an abnormalty exists, or to determine the extent to which abnormalty exists, e.g. the extent or stage of preeclampsia. Comparison to a reference set or profile is particularly useful in applications of the above-described methods, for example, when they are used in methods of diagnosing, prognosing, monitoring, staging and predicting the onset of preeclampsia in a mammal. Accordingly, a method for diagnosing, monitoring, prognosing or staging, or predicting the onset of preeclampsia may comprise: (a) determining the level of expression of one or more preeclampsia biomarkers and b) comparing the level of expression of the preeclampsia biomarkers to a reference set of expression levels of preeclampsia biomarkers, e.g. preeclampsia expression levels that may be associated with various stages of preeclampsia and/or normal levels of the biomarkers from a non-preeclamptic sample, e.g. from a healthy mammal.

A method of prognosing, monitoring or staging preeclampsia in a mammal is also provided in which changes over time in the expression or activity level of one or more preeclampsia biomarkers are monitored in order to monitor the preeclampsia. Thus, determination of the expression level or activity of at least one preeclampsia biomarker in a biological sample obtained from the mammal may be compared to the expression or activity levels in a previously obtained biological sample to monitor preeclampsia in the mammal. Increases or decreases in the expression/activity levels of one or more biomarkers may be indicative of an improvements or progression of the diseases depending on the biomarker, e.g. an increase in the expression of an upregulated biomarker, or a decrease in the expression of a downregulated biomarker is indicative of disease progression, while an increase in the expression of a downregulated biomarker, or decrease in the expression of an upregulated biomarker is indicative of an improvement in the disease.

The present methods may also be useful to monitor a mammal undergoing therapy, and thereby determine whether the therapy is effective. Thus, determination of the expression of a preeclampsia biomarker prior to and throughout treatment will indicate whether the therapy is effective. A showing that the differential expression or activity of the biomarker is decreasing between the preeclampsia levels and the level in a healthy mammal is indicative that the therapy is effective. It may also be useful to utilize the present method to determine whether or not a particular therapy will be effective to treat preeclampsia in a particular mammal by incubating a biological sample of the mammal with a selected therapeutic agent to determine if the agent affects the expression level or activity of one or more preeclamptic biomarkers according to the invention. A determination that the agent does affect biomarker expression or activity in a therapeutic manner, i.e. down-regulates an up-regulated biomarker, or up-regulates a down-regulated biomarker, evidences the utility of the therapeutic agent for that mammal. The method may also be utilized to determine the best therapy for a particular mammal by comparing the effects of a selected number of potential therapies, and selecting the therapy that provides the best results in terms of augmenting or inhibiting the expression or activity of one or more preeclamptic biomarkers.

The comparison of the expression or activity level of one or more preeclamptic biomarkers with a control or reference set may be conducted manually or by automated methods, including for example, by computer systems. In this regard, biomarker expression levels may be obtained and introduced into a computer system for comparison with reference levels contained within the computer in a database. Instructions may be provided to the computer via a user interface that prompts a comparison of the entered data with the reference data in the computer to determine how similar the data entered is to the reference data within the database. Such reference data may include either normal control data, data for preeclamptic biomarker levels, or both. The result of the comparison is then revealed by a computer output in a suitable format including textual or visual output, such as graphical or other.

The expression level or activity of the preeclampsia biomarker in a mammal may be compared to a control level either quantitatively or qualitatively. For example, a qualitative (or unitless) comparison may be carried out by determining whether the level of or activity of the preeclampsia biomarker in a subject is higher, lower, or about the same as a control. Optionally, a quantitative comparison may be used to measure or estimate the magnitude of difference in the level of or activity of the preeclampsia biomarker in a subject as compared to a control, such as, for example, a 2-fold change, a 50% change, etc. For example, a quantitative comparison may be carried out by determining the quantity of a preeclampsia biomarker in the sample of a subject mammal as compared to the quantity in a control sample, wherein the quantity has units attached (such as, for example, mg of protein, volume of a spot/band in a gel, intensity of a spot on a phosphoimager or autoradiogram exposure, diameter of a spot on a chromatography plate, etc.).

In another embodiment, the expression level or activity of a preeclampsia biomarker in a biological sample of a mammal may be used to calculate the physiological concentration of the preeclampsia biomarker in the mammal The physiological concentration of the preeclampsia biomarker in the mammal may then be compared to a level or range determined to be normal, for example, the level determined to exist in a control sample obtained from a healthy mammal that does not have preeclampsia.

In certain embodiments, mammals may be screened for expression levels or activity of preeclampsia biomarkers on a regular basis (or at regular intervals) for purposes of diagnosis of preeclampsia, staging of preeclampsia or to monitor the stage or development of preeclampsia, as a tool for prognosis or to monitor the treatment of preeclampsia. The term “prognosis” refers to a determination of the probable outcome in a mammal with respect to preeclampsia, i.e. the probability of a diagnosis of preeclampsia, the severity of the disease or the probability of recovery therefrom.

The term “staging” refers to determining the degree to which a disease has progressed in a mammal.

In one embodiment, the screening of a pregnant mammal for levels of or activity of preeclampsia biomarkers may be carried out about once every trimester, once every month, once every 3 weeks, once every 2 weeks, once every 10 days, once every week, or about once every 144, 120, 96, 72, 48, 24, or 12 hours. In another embodiment, pregnant mammals may be screened on a regular basis throughout pregnancy or on a regular basis during one or more stages of pregnancy, such as, for example, screening on a regular basis during the first, second and/or third trimesters of pregnancy. In yet another embodiment, mammals may be screened for expression levels or activity of the preeclampsia biomarkers after delivery or termination of a pregnancy in order to identify or monitor diseases or disorders associated with the prior pregnancy.

It may be desirable to monitor symptoms of preeclampsia in addition to monitoring the expression level or activity of the preeclampsia biomarker in a mammal. For example, in a mammal suffering from or susceptible to preeclampsia, it may be desirable to monitor the blood pressure and/or level of total protein in the urine of the mammal. In exemplary embodiments, it may be desirable to determine the blood pressure of a mammal on a regular basis (e.g., hourly, daily, weekly, monthly, etc.). Blood pressure may be monitored using any technique known in the art, such as, for example, a Finometer (TNO Biomedical Instruments, Amsterdam, The Netherlands) or a sphygmometer. Urinary protein excretion may be measured by any method known in the art, including, for example, by spectrophotometric assays using a bicinchoninic acid reagent (Pierce, Rockford, Ill.).

### Arrays

In another embodiment, a method of detecting the level of expression of a preeclampsia biomarker may comprise the use of an array, including either a micro- or macroarray. The array comprises a surface or solid support to which a series of specific reagents are bound which are specific to biomarker targets within a biological sample. Biomarker targets include nucleic acid encoding the biomarker as well as the biomarker itself. Thus, the reagents may comprise nucleic acids or nucleic acid analogues that correspond to a region of a nucleic acid encoding a biomarker (e.g., cDNAs, mRNAs, oligonucleotides), or are otherwise capable of binding to a biomarker-encoding gene. Alternatively, the reagent may be a non-nucleic acid probe suitable to bind to the biomarker itself.

The reagents to be affixed to the arrays are typically polynucleotide probes. These DNAs may be obtained by, e.g., polymerase chain reaction (PCR) amplification of gene segments from genomic DNA, cDNA (e.g., by RT-PCR), or cloned sequences. The probes are chosen, based on the known sequence of the genes or cDNA, which result in binding of unique fragments (e.g., fragments that do not share more than 10 bases of contiguous identical sequence with any other fragment on the microarray) so as to maintain specificity in the array. The probes may also be made from plasmid or phage clones of genes, cDNAs (e.g., expressed sequence tags), or inserts therefrom.

The probes are affixed to the solid support of the array using methods known in the art, for example, as described in Schena et al., 1995, Science 270:467-470; DeRisi et al., 1996, Nature Genetics 14:457-460; and Shalon et al., 1996, Genome Res. 6:639-645, the relevant contents of which are incorporated herein by reference. Another method for making a microarray is by making high-density oligonucleotide arrays as described in U.S. Pat. Nos. 5,578,832; 5,556,752; and 5,510,270, the relevant contents of which are incorporated herein by reference. Other methods for making microarrays, e.g., by masking (Maskos and Southern, 1992, Nuc. Acids Res. 20:1679-1684), may also be used. In principal, any type of array, for example, dot blots on a nylon hybridization membrane (see Sambrook et al., Molecular Cloning—A Laboratory Manual (2nd Ed.), Vol. 1-3, Cold Spring Harbor Laboratory, Cold Spring Harbor, N.Y., 1989), may be used, as will be recognized by those of skill in the art.

The components from a biological sample, e.g. nucleic acids, proteins or other components, to be contacted with and bound to the microarray, are generally labelled with a detectable label, such as a fluorescent label, to permit the ready detection, using appropriate detection techniques, when bound to the array. This label may be added to the sample prior to contact with the array or subsequent to contact with the array. Hybridization or binding and wash conditions are chosen so that the population of labeled component from the sample will specifically hybridize/bind to appropriate sites affixed to the matrix. Methods may be employed to minimize non-specific binding of labeled components to the array. For example, non-specific binding of nucleic acid can be decreased by treating the array with a large quantity of non-specific DNA—a so-called “blocking” step.

As will be appreciated by one of skill in the art, the array may also be a tissue array. For example, paraffin-embedded formalin-fixed specimens may be prepared, and punch “biopsy” cores taken from separate areas of the specimens. Each core may be arrayed into a separate recipient block, and sections cut and processed as previously described, for example, in Konenen, J. et al., Tissue microarrays for high-throughput molecular profiling of tumor specimens, (1987) Nat. Med. 4:844-7 and Chung, G. G. et al., Clin. Cancer Res. (In Press). In another embodiment, the array may be a cell culture pellet microarray.

### Screening for Preclampsia Biomarkers

In another aspect of the invention, a method of identifying preeclampsia biomarkers is provided. The identification of certain biomarkers provides a basis by which to identify additional biomarkers, including for example, naturally-occurring derivatives and metabolites of a known biomarker, as well as biomolecules which are involved in the same metabolic functions as known biomarkers. Thus, the method of screening biomolecules from biological samples of preeclamptic mammals comprises identifying biomolecules which exhibit differential expression and/or activity in comparison to the expression or activity thereof in a healthy mammal. For example, preeclampsia biomarkers according to the invention are involved in fatty acid metabolism. Accordingly, fatty acid metabolites, or fatty acids themselves, may be screened for differential expression/activity in preeclampsia biological samples. As one of skill in the art will appreciate, fatty acids may exist in acid form, in a reduced form as fatty alcohols, or in a conjugated form such as conjugated with glycerol (e.g. tristearoylglycerol) or glycerol phosphate (e.g., glycerophospholipids) or conjugated to other sugars to give sphingoids and sphinganine. Each of these fatty acid forms may be screened for differential expression/activity. In another example, screening of proteins involved in the modulation of oxidative stress may reveal additional proteins that are useful as preeclampsia biomarkers. In a further example, blockage or down regulation of enoyl CoA hydratase and Δ3,5, Δ2,4-dienoyl CoA isomerase leads to accumulation of unsaturated fatty acids. Thus, screening for up-regulated unsaturated fatty acids, and related biomolecules, may also reveal preeclampsia biomarkers. Examples of unsaturated fatty acids include butyric (butanoic acid), caproic (hexanoic acid), caprylic (octanoic acid), capric (decanoic acid), lauric (dodecanoic acid), myristic (tetradecanoic acid), palmitic (hexadecanoic acid), stearic (octadecanoic acid), arachidic (eicosanoic acid), behenic (docosanoic acid), lignoceric (tetracosanoic acid), cerotic (hexacosanoic acid), as well as elevated levels of pristanic acid, phytanic acid, dihydroxycholestanoic acid (DHCA), and trihydroxycholestanoic acid (THCA), each of which represent candidates that may serve as preeclampsia biomarkers.

Any technique known to one of skill in the art for evaluating whether a candidate biomarker is a preeclampsia biomarker may be used to discover additional biomarkers, including the methods described herein. In addition to the proteomic techniques described herein and high resolution, robust, MS-based analyses of potential biomarkers in both preeclamptic and normal biological samples, expression mining methods may be used to identify preeclampsia biomarkers, including Serial Analysis of Gene Expression (SAGE), microarray analysis involving the comparison of preeclamptic tissues versus normal tissues, large scale meta analysis of preeclampsia microarray data, mining EST databases and Massively Parallel Signature Sequencing (MPSS).

### Therapeutic Agent Screening

In a further aspect of the invention, a method of screening for candidate therapeutic compounds for treating preeclampsia is provided. The candidate compounds may be nucleic acids such as antisense nucleic acids, siRNAs or nucleic acid analogs, small molecules, polypeptides, proteins or peptidomimetics. The method includes the step of incubating a biological sample that expresses at least one preeclampsia biomarker with a candidate therapeutic compound under conditions suitable for expression of the biomarker(s). The incubation is conducted for a time period suitable to allow the candidate compound to have an impact on the expression or activity of the biomarker, i.e. modulate the expression or activity thereof, as one of skill will appreciate, if it is capable, or potentially capable, of modulating the expression or activity of the biomarker. Using techniques, such as those previously described, the expression or activity of the biomarker is then determined to ascertain whether the candidate therapeutic modulates, or has the potential to modulate or alter, the expression or activity of the preeclampsia biomarker. Preferably, modulation of the expression or activity of the biomarker is such that the expression or activity thereof is normalized, or approaches the expression or activity of the biomarker in a healthy mammal. Modulation may be determined, or postulated, as previously described by assaying for expression of the biomarker nucleic acid or protein levels, for activity or the biomarker or binding of the candidate with the biomarker or nucleic acid encoding the biomarker. Examples of assays contemplated for use in the present invention include, but are not limited to, competitive binding assay, direct binding assay, two-hybrid assay, cell proliferation assay, kinase assay, phosphatase assay, nuclear hormone translocator assay, fluorescence activated cell screening (FACS) assay, colony-forming/plaque assay, and polymerase chain reaction assay. Such assays are well-known to one of skill in the art and may be adapted to the methods of the present invention with no more than routine experimentation.

All of the above screening methods may be accomplished using a variety of assay formats. In light of the present disclosure, those not expressly described herein will nevertheless be known and comprehended by one of ordinary skill in the art. The assays may identify drugs which are, e.g., either agonists or antagonists, of expression or activity of a preeclampsia biomarker or of a protein:protein or protein-substrate interaction of a preeclampsia biomarker. Assay formats which approximate such conditions as formation of protein complexes or protein-nucleic acid complexes, enzymatic activity, and even specific signaling pathways, may be generated in many different forms, and include but are not limited to assays based on cell-free systems, e.g. purified proteins, plasma or urine samples, or cell lysates, as well as cell-based assays which utilize intact cells.

A determination that the candidate compound modulates the level of expression or activity of the preeclampsia biomarker indicates that the candidate compound may be a therapeutic agent for treating or preventing preeclampsia.

### Kits

The present invention provides kits for practise of the present methods. A kit useful for the diagnosis of preeclampsia in a mammal is provided in one embodiment and comprises at least one reagent that is useful to detect a biomarker that exhibits differential expression or activity in a biological sample from a mammal with preeclampsia in comparison with the expression or activity thereof in a healthy mammal. It will be appreciated by one of skill in the art that such kits are also useful for prognosing or staging with respect to preeclampsia; however, the detection of a change in the differential expression or activity of a biomarker as compared to a previous diagnosis or reference data set is sufficient to provide an indication of the prognosis or stage of preeclampsia.

A kit in accordance with one embodiment may comprise one or more antibodies against a preeclampsia biomarker. In other embodiments, a kit may comprise appropriate reagents for determining the level of protein activity in a biological sample of a mammal to be tested.

In another embodiment, a kit may comprise a microarray comprising probes for preeclampsia biomarker-encoding nucleic acids or biomarker-binding components or a mixture of both. A kit may comprise one or more probes, primers or other binding components for detecting the expression level of a preeclampsia biomarker and/or a solid support on which such probes or binding components are attached and which may be used for detecting expression of a preeclampsia biomarker. A kit may further comprise controls, buffers, and instructions for use.

Kits may also comprise a library of preeclampsia biomarker gene expression levels associated with various stages of preeclamplsia as well as the healthy state, e.g. reference sets. The kits may be useful for identifying mammals that are predisposed to developing preeclampsia, as well as for identifying and validating therapeutics for preeclampsia. In one embodiment, the kit comprises a computer readable medium on which is stored one or more reference sets, or at least values representing reference sets. The kit may comprise expression profile analysis software capable of being loaded into the memory of a computer system.

Kit components may be packaged for either manual or partially or wholly automated practice of the foregoing methods. In other embodiments involving kits, this invention contemplates a kit including compositions of the present invention, and optionally instructions for their use. Such kits may have a variety of uses, including, for example, imaging, diagnosis, prognosis, staging, therapy, and other applications.

### EXEMPLIFICATION

The present invention is further illustrated by the following examples which should not be construed as limiting in any way. The contents of all cited references including literature references, issued patents, published or non published patent applications as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986) (Cold Spring Harbor Laboratory Press, Cold Spring Harbor, N.Y., 1986).

### Experimental Design for the Discovery of Biomarkers for Preeclampsia

### Sample Collection

A tissue repository of 275 clinical preeclamptic and control placental samples was created at the London St Josephs' Hospital and was based on the following inclusion criteria. For a pregnancy to be included in sampling for the preeclamptic tissue repository, the maternal blood pressure had to exceed a systolic measurement of 160 mm Hg or a diastolic measurement of 110 mm Hg, and show at least one of several of secondary Inclusion Criteria. Potential preeclamptic placental samples were rejected from this study if any Exclusion Criteria were met. For the purpose of this study, gestationally age-matched placentas that were used as controls were added to the tissue repository if neither inclusion or exclusion criteria were met, provided that the placental age exceeded 22 weeks gestation.

| Inclusion Criteria | Exclusion Criteria |
|---|---|
| Pregnancies included in the | Placentas were excluded from this |
| preeclamptic placental | study if ANY of the following |
| sampling MUST have met one | exclusion criteria were met in |
| of the following primary | the pregnancy: |
| inclusion criteria: | ROM >18 hours |
| A maternal systolic blood | Evidence of chorioamnionitis |
| pressure >160 mmHg OR | Fetal congenital or genetic anomalies |
| A maternal diastolic blood | Polyhydroamnios |
| pressure >110 mmHg | |
| AND any one of the following | |
| secondary inclusion criteria: | |
| Secondary Inclusion Criteria: | |
| Proteinuria: >3 g in 24 hours | |
| OR >3+ by dipstick. | |
| Platelets: <100 × 10⁹/L | |
| IUGR: EFW <5 percentile | |
| Oliguria: <500 ml in 24 hours | |
| Cerebral or visual disturbances | |
| Severe edema | |
| Epigastric pain | |

Collection of control or preeclamptic placentas meeting the inclusion criteria were sampled in the following manner. Immediately after delivery, 12-one centimeter by one centimeter cubes were excised from the placenta. The sampling grid used for the determination of the twelve collection sites insured an even and representative sampling of each placenta.

Each sample then had the basal plate decidua (BPD) and chorionic plate cut away retaining only the chorionic villi (CV). The twelve CV samples from each placenta were immediately snap frozen in liquid nitrogen and pooled in a sample collection bag to comprise one sample. One third of each of the twelve pieces was removed and pooled for proteomic analysis, while the remaining two thirds was retained for RNA analysis by microarray and reverse transcription-polymerase chain reaction (RT-PCR). Additionally, three samples were removed from each control and preeclamptic placenta for the purpose of histological examination. Preeclamptic and control placentas meeting the above sampling criteria were added to the placental tissue repository.

From the tissue repository, an initial sample set of twenty four placentas was selected for analysis by 2D SDS-PAGE. Since this data set was used for the initial determination of differentially expressed proteins between the control and preeclamptic sample groups, it was termed the “discovery sample set”. An additional sample set was selected from the tissue repository in order to validate proteomic differences obtained through the 2D gel analysis of the “discovery sample set”. This group of samples was termed the “validation sample set”.

### Patient Demographics

Performing proteomic analysis on complex tissues from variable sources requires special attention to sample selection. In this study, the details of each pregnancy were carefully examined in order to ensure the closest possible gestational age and conditions of pregnancy within each test group.

### Experimental Design of the Project

The “discovery sample set” was comprised of twenty four samples selected from the placenta tissue repository. These samples were split into two separate groups, the first of which included twelve placenta samples, all under 28 weeks of age. Six of the placenta samples were selected from preeclamptic pregnancies and the other six selected from gestationally age matched controls. The second group was also comprised of twelve placentas (six PE and 6 controls), but these were from post 28 week deliveries. The cutoff of 28 weeks between the two sample sets was a time point that was set in the clinical environment.

The goal of this study was to examine and compare the proteomes of preeclamptic CV and age matched control CV at both below and above the 28 week cutoff. Hence, the under 28 week and over 28 week samples were analyzed separately. An additional group of placentas were selected for the purpose of validating proteomic differences seen in the 2D gel analysis of the “discovery sample set”. The “validation sample set” was also comprised of two groups. The samples in the first group were all under 28 weeks gestation and were made up of 9 control and 10 preeclamptic samples. The second set was comprised of 10 control and 10 preeclamptic samples, all of which were above 28 weeks gestation. Once differentially regulated proteins were identified in the discovery sample set, the expression levels were validated by western blotting with an independent group of control and preeclamptic samples.

The general extraction procedure involved extraction of proteins from the placentas in each group, desalting by dialysis, followed by determination of the protein concentrations for each sample, and finally analysis of an appropriate amount of protein by 2D SDS-PAGE in duplicate for each sample. All of the gels were imaged and software analyzed for the presence of differentially expressed proteins. Proteins that were determined to be differentially expressed were excised from the gel, digested with trypsin, analyzed by mass spectrometry, and identified through database searching. Proteins that were determined to be differentially regulated were further validated by western blotting.

Although the under and over 28 week placenta sample sets were treated as completely separate experiments, proteins that were identified as being differentially regulated in the under 28 week samples were investigated in the over 28 week samples, and vice versa. By comparing the differentially expressed proteins between the two placental age groups, proteins of interest in each group were assessed with respect to their placental age specificity. This was done in order to determine if the increase or decrease in regulation of a specific protein was the same in the under or over 28 week gestation placental group, or if the expression level of the protein was confined to the gestational age of the placental group examined.

As stated above, the control and preeclamptic CV samples in the “discovery sample set” were split three ways. One third of each CV sample was subjected to 2D SDS-PAGE analysis, one third was subjected to microarray analysis, and the remainder was retained for future experimentation. Handling and treatment of all of the CV samples that were used in this study was done according to the following methodologies.

### Materials and Methods

### Sample Collection of the Chorionic Villi (CV)

Both preeclamptic and control placenta CV samples were collected in a timely manner immediately after delivery. Freshly delivered intact placenta was placed fetal aspect face down and a standardized sample collection grid was superimposed upon the placenta. Individual 1 cm×1 cm tissue samples were excised from the center of each collection grid matrix, giving rise to 12 samples per placenta. The basal plate decidua and the chorionic plate were surgically removed from the CV and each sample was snap frozen in liquid nitrogen. All twelve samples from each individual placenta were pooled and stored at −80° C. for further analysis.

Protein Extraction from the Chorionic Villi

For each individual placental protein extraction performed, a 100 μg portion of each of the 12 samples was chiseled off of the original 1 cm×1 cm samples and pooled to give a sample of ˜1 g (1.00 g-1.29 g). The pooled CV samples remained frozen at all times while being ground under liquid nitrogen with a mortar and pestle. Once the CV was ground to a talcum powder-like consistency, a 200 μg aliquot of this homogenate was removed for protein extraction; the remaining tissue was stored at −80° C. To each 200 μg CV sample was added 435 μL of 2×PE extraction buffer [4% CHAPS (Sigma, St Louis, Mo.), 50 mM ammonium bicarbonate (Sigma), 20 mM DTT (Sigma), 20 μL/mL Ettan protease inhibitor mix (GE Healthcare Bio-Sciences Corp. Piscataway, N.J.), 5 mM EDTA (Fisher Biotech, NJ)]. Protein extraction was allowed to proceed on ice with vigorous shearing of the genomic DNA with a 30.5 gauge needle. To further extract the protein; an aliquot of 380 mg of urea (Sigma) and 139 mg of thiourea (Sigma) was added to the homogenate for a final concentration of 7 M urea, 2 M thiourea. Samples were subsequently dialyzed in Slide-A-Lyzers (Pierce, Rockford, Ill.) against 1 mM EDTA for 48 h. Protein concentrations of the dialyzed samples were determined by Modified Micro-Bradford Assay (Bio-Rad, Hercules, Calif.). Extracted proteins were stored at −80° C. for further analysis.

### Two-Dimensional Polyacrylamide Gel Electrophoresis.

First dimension isoelectric focusing (IEF) was carried out as follows. Sufficient rehydration buffer [6 M urea, 2 M thiourea, 4% CHAPS, 0.4% DTT, 0.5% ampholytes (GE Healthcare), 10 uL/mL Ettan protease inhibitor mix, 10 mM EDTA, 1 mM PMSF(Sigma)] was added to 200 μg of protein to make a total volume of 450 μL. This solution was dispensed evenly across the trough in a reswelling tray and the IPG Immobiline Drystrip pH 3-10 NL (GE Healthcare) was added and covered with mineral oil to prevent evaporation during a 16 h rehydration. The rehydrated strips were focused in an Ettan IPGphor manifold with an Ettan IPGphor II isoelectric focusing apparatus (GE Healthcare). IEF was carried out by using the following voltage gradient: a constant 500 V for 1 h, a 1 h gradient to 1000 V, another 2 h gradient to 5500 V, and finally a constant 5500 V for 8 h, for a total of 99,000 Vh for complete IEF.

For the second dimension, the focused proteins were equilibrated, reduced, and alkylated in SDS equilibration buffer [2% SDS (Sigma), 6 M urea, 30% glycerol (FisherBiotech), 50 mM Tris-Cl pH 8.0 (Bioshop, Burlington, ON)] for 40 min just prior to use. The IPG strips were reduced in 10 mL SDS equilibration buffer with 1% w/v DTT for 20 min at room temperature. The free thiol groups of the proteins' cysteines were then S-carboxyamidomethylated by reaction with 2.5% w/v iodoacetamide (Sigma) for an additional 20 min in 10 mL of SDS equilibration buffer. Polyacrylamide slab gels (25.5 cm×20.5 cm×0.15 cm, 12% acrylamide) were cast in a PROTEAN® Plus Multi-casting chamber (Bio-Rad, Hercules, Calif.). The IPG strips bearing the reduced and alkylated proteins were sealed in place with agarose sealing solution (0.5% Agarose, 0.002% w/v bromophenol blue, 0.1% SDS). The gels were loaded, 12 at a time, into a PROTEAN® Plus Dodeca™ electrophoresis cell (Bio-Rad) and were stacked at a constant 100 V for 1 h. This voltage was finally adjusted to 250 V for 6-7 h, or until the dye front had migrated to within 0.5 cm from the edge of the gel. In the second dimension, the SDS running buffer (25 mM Tris, 192 mM glycine, 0.1% (w/v) SDS, pH 8.3) was held at a constant 15° C. throughout the entire run.

### 2D GE Gel Imaging, Image Analysis and Robotic Spot Picking.

Polyacrylamide gels were stained with the fluorescent stain Sypro Ruby (Molecular Probes, Eugene, Oreg.) for 48 h following overnight fixation in a solution of 50% methanol, 7% acetic acid. In order to minimize background staining, destaining prior to gel image acquisition was carried out for 30 min in a solution of 10% methanol, 7% acetic acid, followed by 3 washes in milliQ dd H₂O. The Sypro Ruby stained gels were imaged on a ProXPRESS Proteomic Imaging System (Perkin-Elmer, Boston, Mass.) using top illumination with a solid black bottom tray and green acrylic sheet for flat field acquisition. The images were acquired at an excitation wavelength of 480 nm and emission wavelength of 620 nm. The scanned gels were analyzed using Phoretix 2D Expressions gel documentation software (Non-Linear Dynamics, Newcastle UK). Differentially regulated spots were selected based on the following criteria: 1) the protein expression between the experimental and control gels had to exceed +/−2-fold regulation to be considered differentially regulated; 2) the spot of interest needed to be present in all of the experimental and control gels; and 3) the spots selected must have been statistically significant as determined by an ANOVA (p-value<0.005). Spots meeting these criteria were excised from the gels by means of robotic spot excision into 96 well plates (Ettan spot picker, GE Healthcare).

### Tryptic Digestion of Protein Samples for Mass Spectrometry

Previously excised 2D SDS-PAGE spots of interest were transferred to 1.5 mL microfuge tubes and destained with alternating washes of 50% (v/v) methanol/5% (v/v) acetic acid and 20% (v/v) acetonitrile/1M ammonium bicarbonate. The destained gel pieces were then dehydrated in 100% acetonitrile, dried in a rotary Speed Vac concentrator (Savant, Hicksville, N.Y.), and reduced for 1 h by rehydration in 30 μL of 10 mM DTT/100 mM ammonium bicarbonate. The free thiol groups of the cysteines were alkylated after removing the DTT solution by aspiration and adding 30 μL of 100 mM iodoacetamide in 100 mM ammonium bicarbonate and incubating for 1 h. The gel pieces were then dried by Speed Vac concentrator and rehydrated with 30 μL of a solution containing 0.6 μg of sequencing grade modified trypsin (Promega, Madison, Wis.) in 50 mM ammonium bicarbonate. After 10 min of rehydration on ice, the excess trypsin solution was removed by aspiration and 5 μl of 50 mM ammonium bicarbonate was added. The tryptic digestion was allowed to proceed at 37° C. for 18 h. The tryptic peptides were extracted from the gel pieces by washing with 30 μl of 50 mM ammonium bicarbonate, followed by two 30 μL washes of 10% (v/v) formic acid, and a final 50 μl, wash with 100% acetonitrile. The washes were pooled in a 500 μL microfuge tube and the volume reduced to dryness in a rotary Speed Vac concentrator (Savant, Hicksville, N.Y.). Samples were stored at −80° C. until analysis.

### Mass Spectrometry

The samples analyzed by MS were brought up in 5% acetonitrile/1% formic acid prior to sample injection. All MS analyses were performed on a Micromass Q-ToF Ultima Global (Waters, Milford, Mass.) running in positive ESI mode. Samples were injected in “μl pick up mode” into a nano-liquid chromatography system comprised of a Micromass Modular CapLC (Waters) using a 5 mm×300 μm 100 Å PepMap C18 p-precolumn (LC Packings, San Francisco, Calif.) and a 15 cm×75 μm 100 Å PepMap C18 column (LC Packings). Peptides were loaded on-column in 0.1% formic acid and eluted into the MS by means of a 5-70% acetonitrile/0.1% formic acid gradient over 60 min. The data-dependent acquisition (DDA) method that was employed followed the following protocol. MS survey scans were run between m/z 400-1800 for 2.4 s and ions exceeding an intensity threshold of 25 counts per second were selected for MS/MS. MS/MS was performed using charge state recognition for the assignment of collision energies on the gated ions for either 10 s or until the TIC rose above 10,000. Upon switching back to the survey scan, previously gated ions were excluded from being re-gated using real time exclusion for a period of 200 s. All samples were run sequentially with identical experimental parameters.

### Database Searching

Protein identifications of the excised protein spots was done using three independent software packages: Protein Lynx Global Server II (Waters), Mascot (Matrix Science Inc, Boston, Mass.), and PEAKS (Bioinformatics Solutions, Waterloo, ON) Positive identification of the spots of interest were based on the following criteria: all three software packages had to arrive at the same protein identification, the spot on the gel had to match both the pI and MW of the putative protein identification, and the MS/MS sequence coverage of the protein had to meet or exceed 10%. If all of these criteria were met, the protein identification was taken as positive.

### Preparation of Additional Preeclamptic and Control Placental Samples for Use in the Validation of Differentially Expressed Proteins.

Additional samples used for the validation of differentially expressed proteins were treated in the following manner. Approximately 50 mg of tissue was removed from each of the twelve 1 cm×1 cm CV samples per placenta and were pooled together. The pooled CV tissue samples obtained from the placental tissue repository ranged from 240-710 mg. To each sample 1 ml of extraction buffer (2% SDS, 50 mM Tris pH 6.8) was added for each 500 mg of CV tissue being extracted. The CV was homogenized at “setting six” using a PowerGen 700 tissue homogenizer (Fisher Scientific). After complete homogenization of the sample, protein extraction was performed by placing the sample in a boiling water bath for 10 minutes. Nucleic acids liberated during cell lysis were sheared through repeated aspirations using a 30.5 gauge needle. After protein extraction and shearing of the nucleic acids, the samples were spun at 15,000×g for 15 minutes at 15° C. to clarify the sample. The protein extracts were aspirated off of the pellet, aliquoted and stored at −80° C. for further use. A 10 μL aliquot was removed from each of the protein extracts to be used in the determination of protein concentration by means of a modified micro-Bradford assay (Bio-Rad).

### Validation of Differentially Expressed Proteins by Immunoblotting.

For validation of the differentially expressed proteins (FABP4 and Per6) in the individual CV samples, 10 g of each placental protein extract described above was resolved by 12% 1D SDS-PAGE and electroblotted onto polyvinylidene fluoride membrane (PVDF) (Bio-Rad Laboratories). Electroblotting was performed at 25 V for 40 min in a Trans-Blot SD semi-dry apparatus (Bio-Rad Laboratories) using Tris/Gly transfer buffer (25 mM Tris, 192 mM glycine, 20% methanol). Blocking of the PVDF membrane was allowed to proceed overnight at room temperature in TBST (10 mM Tris pH 7.6, 150 mM NaCl, 0.1% Tween 20) with 5% skim milk powder. The primary antibodies used were rabbit anti-FABP4 (Cayman, Ann Arbor, Mich.) at a dilution of 1:200, and rabbit anti-peroxiredoxin 6 (Per6) (Abcam, Cambridge, Mass.) at a dilution of 1:2000. For FABP4 and Per6, an HRPO conjugated goat anti-rabbit IgG (Jackson Immunoresearch, West Grove, Pa.) was used as the secondary antibody at a dilution of 1:10,000. All antibodies were diluted in TBST with 5% skim milk powder. After addition of SuperSignal Dura chemiluminescent substrate (Pierce Chemical Company), the immunoblot was developed for 2 min, imaged on a ProXPRESS Proteomic Imaging System (Perkin-Elmer). The immunoreactive protein bands in each of the three immunoblots were quantified using Phoretix 2D Expressions gel documentation software (Non-Linear Dynamics). The loading control for each individual sample was monitored by the quantitation of actin. The primary mouse anti-actin antibody, Ab-5 (Lab Vision, Freemont, Calif.), was used at a concentration of 1:400 and an HRPO conjugated goat anti-mouse IgG (Jackson Immunoresearch) was used as the secondary antibody at a dilution of 1:10,000.

### Experimental Results

### Testing for Differential Analysis Software Fidelity

Successful differential display analysis of the samples used in this study depended on the accurate and reproducible software-based analysis of high quality 2D gels. As each of the CV samples was collected from a different patient, an element of patient-to-patient variability was expected. While variation was not expected in the performance of the 2D gel analysis software, sample-to-sample variability between identical standard samples was determined. These experiments involved analyzing three protein standards, which were run in quadruplicate over a spot concentration range of 50-1000 ng.

In addition to testing for replicate variability during 2D gel analysis with the Phoretix 2D Expressions™ software, the linearity and reproducibility of the software was assessed for the quantitative analysis of artificially “differentially regulated” spots. The same three protein standards that were used to determine the variation among sample replicates were used for this evaluation; the values obtained for the sample replicate assessment were averaged in order to give a value for the spot volume of each concentration tested. For the BSA and myoglobin standards, the 50 ng, 100 ng and 250 ng concentrations were used to represent an artificial differential regulation of 1-, 2- and 5-fold. For the carbonic anhydrase standard the 100 ng, 250 ng, 500 ng, and 1 μg concentrations were used to represent 1-, 2.5-, 5-, and 10-fold differential regulations. For BSA and myoglobin the 50 ng average spot volume was used as the 1:1 ratio reference to which the 2- and 5-fold concentration averages were compared. For carbonic anhydrase, the 100 ng average spot volume was used as the 1:1 ratio reference to which the 2.5-, 5-, and 10-fold concentration averages were compared. The replicate reproducibility and ratio correlation determined for this set of standards correlated with current published data [68].

### Image Analysis of the Experimental Samples

As a general rule, 2D gel analysis using the Phoretix 2D Expressions™ software adheres to the following path of analytical functions. Spots are detected in the gels, noise spots are removed, the background is subtracted from each spot, the images are warped to a master reference gel, spots between all of the gels are matched, individual gels are normalized and comparisons are made for differentially expressed proteins. Additional manual manipulations are occasionally required.

Overall, fewer proteins were detected in the initial survey of the “over-28 weeks” experiment than in the “under-28 weeks” experiment. A possible reason for this is the rapid growth and metabolism of “under 28-week” placental samples as opposed to the more developed “over 28-week” placental samples. The rapid turn-over and varied expression of proteins in the under 28-week samples resulted in an increase of statistically differentially regulated proteins in this experimental group

To describe a simple case, the under-28 week control group will be considered here in describing the preparation of the under-28 week control averaged gel. A single gel in the rank of the under-28 week control sample replicates is selected as the “base gel” for the gel average for this group. After all of the spots are detected and matched within this group, spots are added to the computer-based average gel based on predetermined averaging parameters. In this case, spots had to be present in at least half of the sample replicates to be included in the average gel. Once the control- and preeclamptic-averaged gels were compiled, the software used these spot catalogues to match proteins between the control and preeclamptic spot data sets. Proteins that were present only in the control average but not the preeclamptic average (or vice versa) were not included as matches and were examined independently. This resulted in a lower number of spots being matched between the gels compared to the total number of spots detected. After a closer examination of the spots that were not matched between the control and preeclamptic averages, it was determined that they were not statistically significant, due to extremely poor reproducibility and significant variation.

Upon further examination of the matched data between the control and preeclamptic averages, differentially regulated spot lists were extracted and subjected to further analysis. Each of the spots that were suspected as being differentially regulated was manually examined for presence in all of the gels within the experiment to confirm whether successful spot matching had occurred. After excluding spots that were not correctly matched, that were statistically insignificant in terms of differential expression (an ANOVA p-value of >0.05), or that were so complex that accurate spot values were impossible to obtain, a substantially smaller, but highly significant data set, was arrived at. The final numbers obtained from this filtering yielded two up-regulated and seven down-regulated proteins in the preeclamptic data set in the under-28 week experiment, and three up-regulated and zero down-regulated proteins in the preeclamptic data set in the over-28 week experiment.

### Experimental Differential Regulation Analysis Results

After confirming the reproducibility and fidelity of the gel analysis software, both the under-28 week and over-28 week CV experimental groups were analyzed. The less than 28 week CV samples were analyzed with the Phoretix software using the previously stated parameters. Differentially regulated spots that were reproducibly observed in both the control and preeclamptic CV samples were excised and subjected to MS analysis.

After extensive 2D gel analysis, nine proteins were identified reproducibly as being differentially regulated between the control and preeclamptic CV samples. Each of these proteins was excised and successfully identified by LC-MS/MS. Table I summarizes the differentially expressed proteins identified, the average normalized spot volumes of each identified protein, the statistical significance of the spot differences and the overall regulation difference between the under-28 week control and preeclamptic samples.

TABLE I
Protein Identifications in the under-28 week analysis
of five control and five preeclamptic pregnancies.

| | Control | Preeclamptic | | |
|---|---|---|---|---|
| | Pregnancy | Pregnancy | Fold | |
| | Average Spot | Average Spot | Difference | |
| | Volumes* | Volumes* | PE vs. | ANOVA |
| Proteins Identified | (n = 5) | (n = 5) | Control | p-value |
| Fatty Acid Binding Protein 4 | 0.057 (0.049) | 0.213 (0.074) | 3.7 | <0.0001 |
| Peroxiredoxin 6 | 0.093 (0.028) | 0.250 (0.057) | 2.7 | <0.0001 |
| Enoyl CoA Hydratase | 0.116 (0.038) | 0.060 (0.025) | −2 | <0.005 |
| Estradiol 17-beta- | 0.197 (0.050) | 0.096 (0.031) | −2 | <0.0001 |
| dehydrogenase | | | | |
| Stathmin | 0.080 (0.018) | 0.038 (0.021) | −2.1 | <0.0001 |
| Human Placental Lactogen | 0.239 (0.057) | 0.097 (0.043) | −2.5 | <0.0001 |
| Lipocortin | 0.063 (0.020) | 0.026 (0.009) | −2.4 | <0.0001 |
| Proliferation-associated | 0.052 (0.017) | 0.019 (0.007) | −2.7 | <0.001 |
| protein 2G4 | | | | |
| Δ3,5-Δ2,4-dienoyl-CoA | 0.132 (0.085) | 0.038 (0.014) | −3.4 | <0.005 |
| isomerase | | | | |

The values for spot volumes are the averages of the five different CV samples run in duplicate (ten gels total). Ten values were averaged for the control samples and ten values averaged for the preeclamptic samples. Calculated standard deviations are shown in brackets for each average. Fold differences are the preeclamptic averages divided by the control averages.
*values displayed as: normalized spot volume (standard deviation)

Each of the proteins that were identified in the under-28 week samples complied with the isoelectric point, molecular weight, MS/MS minimum peptide cutoff, and total protein coverage for the MS identification criteria previously described. The details of the MS/MS and observed frequencies of each protein of interest amongst the samples tested are summarized in Table II. Each of the proteins of interest had acceptable MS/MS coverage with adequate numbers of observed peptides with which to base the protein identifications. Additionally, each differentially regulated protein of interest was observed in each of the CV samples tested.

TABLE II
Protein Identification data for the under-28 week analysis.

| | | | | Number of |
|---|---|---|---|---|
| | Number of | | Total | Gels containing |
| Protein | Peptides | Mascot | Protein | the Protein of |
| Identification | observed | Score | coverage | Interest |
| Fatty Acid Binding | 6 | 374 | 48% | 10/10 |
| Protein 4 | | | | |
| Peroxiredoxin 6 | 9 | 416 | 52% | 10/10 |
| Enoyl-CoA Hydratase | 7 | 345 | 35% | 10/10 |
| Estradiol 17-beta- | 14 | 818 | 53% | 10/10 |
| dehydrogenase | | | | |
| Stathmin | 4 | 169 | 28% | 10/10 |
| Human Placental | 5 | 316 | 29% | 10/10 |
| Lactogen | | | | |
| Lipocortin | 6 | 437 | 21% | 10/10 |
| Proliferation- | 5 | 186 | 12% | 10/10 |
| associated | | | | |
| protein 2G4 | | | | |
| Δ3,5-Δ2,4-dienoyl- | 12 | 564 | 39% | 10/10 |
| CoA isomerase | | | | |

Each protein identified was present in all of the gels that were tested. LC-MS/MS yielded adequate numbers of peptides and sufficient coverage giving high confidence identifications for each protein analyzed.

The over-28 week control and preeclamptic CV samples were analyzed with an identical protocol as the under-28 week samples. Table III summarizes the three proteins that were reproducibly and statistically determined to be differentially regulated in the over-28 week samples. As per the under-28 week samples, the average normalized spot volume of each protein identified, the statistical significance of the spot differences, and the overall regulation difference between the control and preeclamptic samples are summarized.

TABLE III
Protein Identifications in the over-28 week analysis
of five control and six preeclamptic pregnancies.

| | Control | Preeclamptic | | |
|---|---|---|---|---|
| | Pregnancy | Pregnancy | Fold | |
| | Average Spot | Average Spot | Difference | |
| | Volumes* | Volumes* | PE vs. | ANOVA |
| Protein Identification | (n = 6) | (n = 5) | Control | p-value |
| Macrophage capping | 0.020 (0.013) | 0.044 (0.014) | 2.2 | <0.0005 |
| protein | | | | |
| Heat shock protein β-1 | 0.064 (0.021) | 0.129 (0.060) | 2 | <0.005 |
| Prostaglandin | 0.053 (0.029) | 0.110 (0.035) | 2 | <0.0005 |
| dehydrogenase 1 | | | | |

The values for spot volumes are the averages of the five different CV samples in duplicate. Ten values were averaged for the control samples and ten values averaged for the preeclamptic samples. Calculated standard deviations are shown in brackets for each average. Fold differences are the preeclamptic averages divided by the control averages.
*values displayed as: normalized spot volume (standard deviation)

All three proteins of interest in the over-28 week samples followed the same MS-based identification criteria as the under-28 week samples. The over-28 week MS/MS results are shown in Table IV and highlight the number of peptides seen, total MS/MS protein coverage, and observation frequency of each protein between the different samples of the over-28 week experiment. In general, there were far fewer proteins that were observed to be differentially regulated in the over-28 week sample set than in the under-28 week sample set.

TABLE IV
Protein Identification data for the over-28 week analysis.

| | | | | Number of |
|---|---|---|---|---|
| | Number of | | Total | Gels containing |
| Protein | Peptides | Mascot | Protein | the Protein of |
| Identification | Observed | Score | coverage | Interest |
| Macrophage | 5 | 242 | 18% | 11/11 |
| capping protein | | | | |
| Heat shock | 6 | 172 | 35% | 11/11 |
| protein β-1 | | | | |
| Prostaglandin | 6 | 535 | 42% | 11/11 |
| dehydrogenase 1 | | | | |

Each protein identified was present in all of the gels that were tested. The LC-MS/MS analyses yielded adequate numbers of peptides and sufficient coverage giving high confidence identifications for each protein analyzed.

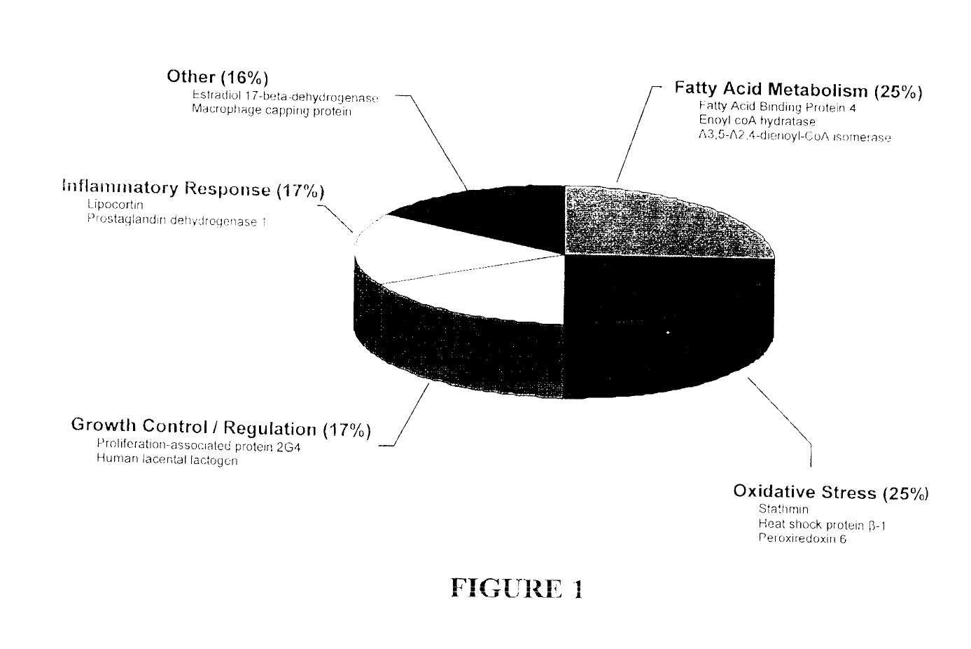

Selecting and identifying the proteins that were determined to be differentially regulated yielded valuable information about proteins involved in the underlying process of preeclampsia. However, further validation of these results was required. After careful examination of the proteins that were determined to be differentially regulated in this study, several functional groups were identified. Functional groupings were assigned using the gene ontology (GO) database and GoMiner™, a comprehensive tool for the analysis of protein function as described in Zeeberg et al. (Genome Biol, 2003. 4(4): p. R28), the contents of which are incorporated herein by reference. After GoMiner™ analysis of the under and over-28 week data sets, differentially expressed proteins were functionally classified into the groups shown in FIG. 1.

### Validation of Differentially Regulated Spots by Western Blotting

Because of the large number of proteins that were determined to be differentially expressed, only a few proteins were selected for further validation of differential expression. Although there are many ways in which the expression levels of these proteins could be validated, expression validation by western blotting was used in this study.

As mentioned previously there were two data sets used in this study. The first data set, termed the discovery data set, was used for the initial 2D analysis of the control and preeclamptic CV samples. These samples were employed to identify any differentially regulated proteins present. The second data set was not subjected to 2D gel analysis, but was instead used as a validation set to confirm the differential expression levels identified in the discovery data set. This secondary data set was termed the “validation data set”.

For reasons discussed below, only two of the proteins showing differential regulation were subjected to analysis by western blotting in both the discovery and validation data set. One of the proteins that was assayed by western blotting is involved in fatty acid metabolism, while the other was involved in the mediation of oxidative stress. The fatty acid metabolism-related protein that was validated was fatty acid binding protein 4 (FABP4), and the protein involved in the mediation of oxidative stress was peroxiredoxin 6 (Per6). Initially the expression levels of these two proteins were validated in the discovery data set to confirm and support the differences in regulation seen in the 2D gel analysis.

It is clear that a substantial difference in FABP4 expression exists between the under-28 week control and preeclamptic CV samples. This difference does not exist in the over-28 week samples, and this points to a proteomic difference confined to early preeclamptic pregnancies. After the actin loading controls were taken into account and the expression values normalized and quantified using Phoretix software, it was found that FABP4 expression was increased by a factor of 8.49 (ANOVA p-value=<0.0001) in the under-28 week preeclamptic samples and by a factor of 1.06 (ANOVA p-value=>0.83) in the over-28 week preeclamptic samples.

Although the differences in protein expression for Per6 are not as striking as with FABP4, there was still a statistical difference between the control and preeclamptic CV samples. Similar to the FABP4, the Per6 expression level only differed in the under-28 week samples and not in the over-28 week samples. After normalization of the samples to the actin loading controls, it was found that Per6 expression was increased in the under-28 week preeclamptic samples by 2.79 fold (ANOVA p-value=<0.01) and marginally decreased 0.69 fold (ANOVA p-value=>0.1) in the over-28 week samples.

The discovery data set was comprised of 10 under-28 week CV samples and 12 over-28 week CV samples. While both data sets were evenly divided between control and preeclamptic conditions, the significance of the differentially expressed proteins identified required further validation using a new set of samples. The validation data set was selected from patient samples in the placental tissue repository that were different from those of the discovery data set. This data set was comprised of 18 CV samples of under-28 weeks gestation and 20 CV samples of over-28 weeks gestation. Like the discovery data set, each of these groups were evenly divided between control and preeclamptic experimental conditions.

The western blot results of the validation data set were very similar to that of the discovery data set, and this served to confirm the expression values observed in the 2D gel analysis. As with the discovery data set validation western blots, actin was used as the loading control for the validation data set. Once the loading controls were used to normalize the expression levels of FABP4, it was found that FABP4 expression was increased in the preeclamptic samples by 4.59 fold (ANOVA p-value=<0.001) in the under-28 week data set and by 1.13 fold (ANOVA p-value=>0.35) in the over-28 week data set. In terms of Per6 expression levels, after normalization using the actin loading control, it was found that there was a 2.05 fold increase (ANOVA p-value=<0.001) in expression in the under-28 weeks gestation preeclamptic samples and a 0.88 fold decrease (ANOVA p-value=>0.39) in expression in the over-28 weeks gestation preeclamptic samples.

The up-regulation of FABP4 in early preeclamptic samples was very similar between both the discovery and validation data sets. Due to both the limited number of samples analyzed and the inherent patient-to-patient diversity of placental tissue samples in this study, the standard deviations of each expression level reflect this level of expected variation. Since many proteins related to pregnancy are regulated in a gestational age related manner, the variation seen amongst different samples within each group may be due to the variety of different gestational ages in both the under- and over-28 week sample sets. Every effort was taken to maintain the closest possible ranges in the gestational age of the placentas during sample selection. Despite considerable efforts in the selection and analysis of the sample sets, there remained notable variations in the regulations of the proteins that were identified as being differentially expressed in the sample sets. Although there were notable standard deviations associated with the results, it should be noted that the ANOVA scores summarized in Tables V and VI showed that in the under-28 week samples, the regulation of both FABP4 and Per6 between the control and preeclamptic samples were statistically significant events. In terms of the over-28 week FABP4 and Per6 expression levels, it can be seen that no statistically significant differences existed as shown by the extremely high ANOVA p-values shown in Tables V and VI.

TABLE V
Total regulation statistics for the fatty acid binding
protein (FABP4) discovery and validation data sets.
FABP4 Discovery Data Set

| | Under-28 weeks | | Over-28 weeks | |
|---|---|---|---|---|
| | Control | PE | Control | PE |
| | n = 5 | n = 5 | n = 6 | n = 6 |
| Group Averages | 5097526 | 43299122 | 23156783 | 24705966 |
| Standard Deviations | 3624230 | 11451548 | 13833129 | 11174454 |

| | | |
|---|---|---|
| Differential | 8.49 +/− 6.44 | 1.06 +/− 0.79 |
| Regulation Values | ANOVA | ANOVA |
| | p-value = <0.0001 | p-value = >0.83 |

FABP4 Validation Data Set

| | Under-28 weeks | | Over-28 weeks | |
|---|---|---|---|---|
| | Control | PE | Control | PE |
| | n = 9 | n = 9 | n = 10 | n = 10 |
| Group Averages | 6347268 | 29147290 | 24438689 | 27792686 |
| Standard Deviations | 8105040 | 14246549 | 6510942 | 9234250 |

| | | |
|---|---|---|
| Differential | 4.59 +/− 6.27 | 1.13 +/− 0.48 |
| Regulation Values | ANOVA | ANOVA |
| | p-value = <0.001 | p-value = >0.35 |

Values shown are the normalized spot volume averages for each group as well as the associated standard deviation. Differential regulation values are the average fold difference in the preeclamptic data sets +/− standard deviations. One way ANOVA values are shown to highlight statistically relevant differences.

TABLE VI
Total regulation statistics for the peroxiredoxin
6 (Per6) discovery and validation data sets.
Per6 Discovery Data Set

| | Under-28 weeks | | Over-28 weeks | |
|---|---|---|---|---|
| | Control | PE | Control | PE |
| | n = 5 | n = 5 | n = 6 | n = 6 |
| Group Averages | 3991892 | 11163517 | 8939211 | 6232705 |
| Standard Deviations | 1440004 | 5222873 | 3122789 | 1815760 |

| | | |
|---|---|---|
| Differential | 2.79 +/− 1.65 | 0.69 +/− 0.44 |
| Regulation Values | ANOVA | ANOVA |
| | p-value = <0.01 | p-value = >0.1 |

Per6 Validation Data Set

| | Under-28 weeks | | Over-28 weeks | |
|---|---|---|---|---|
| | Control | PE | Control | PE |
| | n = 9 | n = 9 | n = 10 | n = 10 |
| Group Averages | 10095459 | 20704498 | 10810546 | 9574273 |
| Standard Deviations | 4679979 | 4949433 | 3605970 | 2400218 |

| | | |
|---|---|---|
| Differential | 2.05 +/− 1.38 | 0.88 +/− 0.53 |
| Regulation Values | ANOVA | ANOVA |
| | p-value = <0.001 | p-value = >0.39 |

Values shown are the normalized spot volume averages for each group as well as the associated standard deviation. Differential regulation values are the average fold difference in the preeclamptic data sets +/− standard deviations. One way ANOVA values are shown to highlight statistically relevant differences.

### Discussion

Several proteins were identified with statistical significance as biomarkers of preeclampsia by their differential regulation. These proteins can be divided into five functional groups, including fatty acid metabolism-related proteins, oxidative stress-related proteins, proteins involved with growth regulation and control and proteins involved in the inflammatory response. The majority of the biomarker candidates identified were linked to fatty acid metabolism, or cellular mediators of oxidative stress.

Several of the differentially regulated proteins identified in this study are directly involved in the metabolism of fatty acids: fatty acid binding protein 4 (FABP4), enoyl-CoA hydratase (ECHS1), and Δ3,5-Δ2,4-dienoyl-CoA isomerase (ECH1). As shown above, FABP4 was validated by western blotting as being differentially expressed in preeclamptic pregnancies. As determined by 2D SDS-PAGE, there was a 3.7-fold up-regulation of this protein in the preeclamptic CV samples. The proteins, ECHS1 and ECH1, were both determined to be down-regulated in preeclampsia by 2.0- and 3.4-fold, respectively. Both of these proteins are key enzymes required for the f3-oxidation of fatty acids.

In this study, differentially regulated proteins related to oxidative stress were identified. Of these proteins, the antioxidant protein Per6 and heat shock protein β-1 (HSP27), which are involved in the mediation of oxidative stress, were up-regulated in the preeclamptic samples by factors of 2.7 and 2-fold, respectively. Another protein, stathmin, a microtubule destabilizing protein, was down-regulated by a factor of 2.1 in preeclampsia, and has been found in other studies to be down-regulated in response to oxidative damage.

Proteins involved in the inflammatory response that were found to be differentially regulated in the preeclamptic CV samples were lipocortin (LPC1) and prostaglandin dehydrogenase 1 (PGDH1). In this study, it was determined that PGDH1 was up-regulated by a factor of 2 in the preeclamptic samples. In contrast to the up-regulation of PDGH1, LPC1 was determined to be down-regulated by a factor of 2.4 in the preeclamptic samples.

Additional proteins that are differentially expressed in preeclampsia are proliferation-associated protein 2G4 (PA2G4) and estradiol 17-beta-dehydrogenase (HSD17B1). Both of these proteins perform functions that are related to the metabolism of estrogen precursors. PA2G4 is also known to function as a growth regulatory molecule In this study, it was found that PA2G4 was down-regulated by 2.7-fold in the preeclamptic samples, which could indicate the potential down-regulation of estrogen precursor transcription in preeclamptic pregnancies. In this study, it was also noted that HSD17B1, a protein responsible for the conversion of estrogen precursors to estradiol, was down-regulated by 2.0-fold in the preeclamptic samples.

The proteins, human placental growth hormone, or chorionic sommatomammotropin hormone (CSH1), and macrophage capping protein (CAPG), were also identified as regulated in preeclampsia. A 2.5-fold down-regulation of CSH1 was identified in this study. CAPG was identified as being up-regulated in preeclampsia by a factor of 2.2.

FIG. 1 illustrates the proteins identified in this study grouped into functional categories. The identification of proteins belonging to similar metabolic processes provides a basis for further preeclamptic biomarker discovery.

The identification of differentially regulated proteins in preeclampsia in the under-28 week samples advantageously provides a means of early detection of preeclampsia.

In this study, differentially proteins were identified in both the under-28 week data set and the over-28 week data set. There was no overlap in the identity of proteins determined to be differentially regulated between these two data sets which indicates that the protein biomarkers were age-dependent and related to specific biological processes that were occurring within a specific gestational age window.

1. A method for diagnosing, detecting or predicting the onset of preeclampsia in a mammal, comprising the steps of:
(a) determining in a biological sample from the mammal the expression level of or activity of at least one preeclampsia biomarker, and
(b) comparing the expression level or activity of each preeclampsia biomarker with the level of or activity of the biomarker in a healthy mammal free of preeclampsia, wherein a differential expression level or differential activity of the biomarker is indicative of preeclampsia in the mammal, or that preeclampsia may develop in the mammal.

2. The method as defined in claim 1, wherein at least one of said biomarkers is a protein.

3. The method as defined in claim 1, wherein at least one of said biomarkers is involved in the metabolism of fatty acids:

4. The method as defined in claim 3, wherein said biomarker is selected from the group consisting of fatty acid binding protein 4 (FABP4), enoyl-CoA hydratase (ECHS1), and Δ3,5-Δ2,4-dienoyl-CoA isomerase (ECH1).

5. The method as defined in claim 1, wherein at least one of said biomarkers is involved in oxidative stress.

6. The method as defined in claim 5, wherein said biomarker is selected from the group consisting of Per6, heat shock protein β-1 (HSP27) and stathmin.

7. (canceled)

8. (canceled)

9. The method defined in claim 1, wherein the preeclampsia biomarker is selected from the group consisting of FABP4, ECHS1, ECH1, antoxidant protein Per6, heat shock protein ∃-1, stathmin, lipocortin, prostaglandin dehydrogenase 1, proliferation-associated protein 2G4, placental growth hormone (chorionic sommatomammotropin hormone (CSH1), estradiol 17-beta dehydrogenase, and macrophage capping protein.

10. The method defined in claim 1, wherein the biomarker is a fatty acid or a derivative or metabolite thereof.

11. The method of claim 10, wherein the fatty acid is selected from the group consisting of butanoic acid, hexanoic acid, octanoic acid, decanoic acid, dodecanoic acid, tetradecanoic acid, hexadecanoic acid, octadecanoic acid, eicosanoic acid, docosanoic acid, tetracosanoic acid, hexacosanoic acid, pristanic acid, phytanic acid, dihydroxycholestanoic acid (DHCA) and trihydroxycholestanoic acid (THCA).

12. (canceled)

13. (canceled)

14. (canceled)

15. A method of prognosing, monitoring or staging preeclampsia in a mammal comprising the steps of:
(a) determining in a first biological sample from the mammal the expression level or activity of at least one preeclampsia biomarker, and
(b) comparing the expression level or activity of each preeclampsia biomarker with the level or activity of the biomarker in the mammal as determined in a second biological sample obtained prior to the first sample to monitor the preeclampsia in the mammal.

16. The method as defined in claim 15, wherein at least one of said biomarkers is involved in the metabolism of fatty acids:

17. The method as defined in claim 15, wherein said biomarker is selected from the group consisting of fatty acid binding protein 4 (FABP4), enoyl-CoA hydratase (ECHS1), and Δ3,5-Δ2,4-dienoyl-CoA isomerase (ECH1).

18. The method as defined in claim 15, wherein at least one of said biomarkers is involved in oxidative stress.

19. The method as defined in claim 18, wherein said biomarker is selected from the group consisting of Per6, heat shock protein β-1 (HSP27) and stathmin.

20. The method defined in claim 15, wherein the preeclampsia biomarker is selected from the group consisting of FABP4, ECHS1, ECH1, antoxidant protein Per6, heat shock protein ∃-1, stathmin, lipocortin, prostaglandin dehydrogenase 1, proliferation-associated protein 2G4, placental growth hormone (chorionic sommatomammotropin hormone (CSH1), estradiol 17-beta dehydrogenase, and macrophage capping protein.

21. A microarray for use in diagnosing preeclampsia in a mammal as defined in claim 1, comprising one or more reagents capable of detecting the level of expression or activity of at least two preeclampsia biomarkers in a biological sample of the mammal, wherein the biomarkers exhibit a differential expression or activity in comparison to the expression or activity thereof in a healthy mammal and wherein the reagents are attached to a solid support.

22. A microarray as defined in claim 21, wherein the preeclampsia biomarker is selected from the group consisting of FABP4, ECHS1, ECH1, antoxidant protein Per6, heat shock protein ∃-1, stathmin, lipocortin, prostaglandin dehydrogenase 1, proliferation-associated protein 2G4, placental growth hormone (chorionic sommatomammotropin hormone (CSH1), estradiol 17-beta dehydrogenase, and macrophage capping protein.

23. A method of screening candidate therapeutic compounds for treating preeclampsia in a mammal comprising the steps of:
(a) incubating a biological sample that expresses at least one preeclampsia biomarker with said candidate therapeutic, and
(b) determining whether said candidate therapeutic modulates the expression of or activity of the preeclampsia biomarker, wherein modulation of the level of expression or activity of the preeclampsia biomarker indicates that the candidate therapeutic may be a therapeutic agent for treating or preventing preeclampsia.

24. A kit useful in a method of diagnosing preeclampsia in a mammal as defined in claim 1, said kit comprising instructions and at least one reagent that is useful to detect at least one biomarker selected from the group consisting of FABP4, ECHS1, ECH1, antoxidant protein Per6, heat shock protein ∃-1, stathmin, lipocortin, prostaglandin dehydrogenase 1, proliferation-associated protein 2G4, placental growth hormone (chorionic sommatomammotropin hormone (CSH1), estradiol 17-beta dehydrogenase, and macrophage capping protein.

25. (canceled)

26. (canceled)

27. (canceled)

28. A method as defined in claim 15, for monitoring the treatment of preeclampsia in a mammal, wherein the expression level or activity the preeclampsia biomarker in the first and second biological samples taken at different time points during treatment is additionally compared to the expression level or activity of the preeclampsia biomarker in a healthy mammal, wherein a reduced differential expression level or differential activity of the biomarker in a biological sample as compared with the healthy mammal is indicative that the treatment is effective.
